(19) United States
(12) Patent Application Publication
(10) Pub. No.: US 2010/0324345 A1
(43) Pub. Date: Dec. 23, 2010

(54) SYSTEMS AND PROCESSES FOR CFO-1113 FORMATION FROM HCFC-123a

(75) Inventors: Selma Bektesevic, Williamsville, NY (US); Hsueh Sung Harry Tung, Getzville, NY (US); Haluk Kopkalli, Staten Island, NY (US)

Correspondence Address: HONEYWELL INTERNATIONAL INC.;PATENT SERVICES, 101 COLUMBIA ROAD, P O BOX 2245, MORRISTOWN, NJ, 07962-2245, US

(73) Assignee: Honeywell International Inc., Morristown, NJ (US)

(21) Appl. No.: 12/488,684

(22) Filed: Jun. 22, 2009

(51) Int. Cl. C07C 17/25

(52) U.S. Cl. 570/156; 570/155

(57) ABSTRACT

Systems and processes relating to the formation and production of CFO-1113 HCFC-123a. Such systems and processes can include one or more reactors in series that react HCFC-123a and base to produce reaction product vapors including CFO-1113. Optionally, a phase transfer agent or catalyst can be added to the reaction to enhance the reaction rate. The CFO-1113 can be separated from the reaction product vapors to produce a CFO-1113 product stream. The reactions can be conducted continuously, and a liquid effluent stream can be removed from the reactors during the reaction. Unreacted HCFC-123a can be separated from the liquid effluent stream and provided back to the reactors.

### FIELD OF THE INVENTION

The systems and processes described herein relate to the formation and production of chlorotrifluoroethylene (CFO-1113 or CTFE), and more particularly to the production of CFO-1113 from HCFC-123a.

### DESCRIPTION OF RELATED ART

Chlorotrifluoroethylene, which is CF₂═CFCl, and is often referred to as CFO-1113 or CTFE, is a monomer utilized in the field of fluororesins and fluorine rubbers. For example, CFO-1113 can be utilized in the production of polychlorotrifluoroethylene, as well as in the production of various copolymers with ethylene, vinyl acetate or vinylidene fluoride, among others.

Commonly, CFO-1113 is manufactured by dechlorination of 1,1,2-trichloro-1,2,2-trifluoroethane (CFC-113). In such a reaction, CFC-113 and zinc (Zn) can be reacted in the presence of methanol to yield CFO-1113. High conversions and selectivities to CFO-1113 can be obtained in such reactions, although some by-products are also formed, including 1,2-dichloro-1,1,2-trifluoroethane (CHClF—CClF₂ or HCFC-123a), trifluoroethene (HFC-1123), 1-chloro-2,2,2-trifluoroethane (CFC-133a), unreacted CFC-113, and methanol.

### SUMMARY OF THE INVENTION

The systems and processes described herein relate to the formation and production of CFO-1113 from HCFC-123a. More particularly, the systems and processes described herein relate to the formation and production of CFO-1113 directly from HCFC-123a.

In one aspect, a system for producing CFO-1113 from HCFC-123a is provided that includes at least one reactor, a condenser, and a phase separator. The reactor can receive an HCFC-123a containing feed stream and a base containing feed stream. The HCFC-123a and the base can be reacted in the at least one reactor to produce reaction product vapors including CFO-1113, and a liquid effluent stream containing unreacted HCFC-123a can be removed from the at least one reactor. The condenser can receive reaction product vapors from the reactor and produces a CFO-1113 product stream. The phase separator can receive liquid effluent stream from the reactor and separate unreacted HCFC-123a to produce an unreacted HCFC-123a stream. The unreacted HCFC-123a stream can be recycled for further reaction.

In another aspect, a process for producing CFO-1113 from HCFC-123a is provided. The process includes providing a first reactor, providing an HCFC-123a containing feed stream to the first reactor, and providing a base containing feed stream to the first reactor. The process also includes, reacting the HCFC-123a and the base in the first reactor to produce reaction product vapors including CFO-1113. The process further includes removing the reaction product vapors from the first reactor, and removing a liquid effluent stream from the first reactor, where the liquid effluent stream contains base and unreacted HCFC-123a.

In a third aspect, another process for producing CFO-1113 from HCFC-123a is provided. The process includes providing a first reactor, providing an HCFC-123a containing feed stream to the first reactor, and providing a base containing feed stream to the first reactor. The process includes reacting the HCFC-123a and the base in the first reactor to produce reaction product vapors including CFO-1113, removing the reaction product vapors from the first reactor, and removing a liquid effluent stream from the first reactor, where the liquid effluent stream contains base and unreacted HCFC-123a. The process also includes providing a second reactor, providing the liquid effluent stream containing base and unreacted HCFC-123a from the first reactor to the second reactor, optionally providing additional HCFC-123a to the second reactor, and reacting the HCFC-123a and the base in the second reactor to produce reaction product vapors including CFO-1113. The process further includes removing the reaction product vapors from the second reactor, and removing a liquid effluent stream from the second reactor, where the liquid effluent stream contains base and unreacted HCFC-123a.

### BRIEF DESCRIPTION OF THE DRAWINGS

Specific examples have been chosen for purposes of illustration and description, and are shown in the accompanying drawings, forming a part of the specification.

FIG. 1 illustrates a system for the process of producing CFO-1113 from HCFC-123a including a single reactor.

FIG. 2 illustrates a system for the process of producing CFO-1113 from HCFC-123a including two reactors.

### DETAILED DESCRIPTION

The systems and processes described herein relate to the formation and production of CFO-1113. More particularly, the systems and processes described herein relate to the formation and production of CFO-1113 from HCFC-123a.

As described above HCFC-123a is a side product in the manufacture of CFO-1113 from the reaction of CFC-113 and zinc in the presence of methanol. It can thus be desirable to have a process wherein HCFC-123a is converted to useful products, preferably CFO-1113, which could increase overall process yield in such CFO-1113 production processes. The systems and processes described herein preferably derive CFO-1113 from HCFC-123a through a one step, or direct, reaction process.

For example, HCFC-123a can be reacted with a suitable base to directly form CFO-1113 and other byproducts. Suitable bases include, but are not limited to, potassium hydroxide (KOH), sodium hydroxide (NaOH), calcium oxide (CaO), and calcium hydroxide (Ca(OH)₂).

HCFC-123a can be combined with potassium hydroxide (KOH) to form CFO-1113, potassium chloride (KCl), and water, as shown in reaction (1) below:

CHClF—CClF₂+KOH→CF₂═CFCl+KCl+H₂O (1)

HCFC-123a can be combined with sodium hydroxide (NaOH) to form CFO-1113, sodium chloride and water, as shown in reaction (2) below:

CHClF—CClF₂+NaOH→CF₂═CFCl+NaCl+H₂O (2)

HCFC-123a can be combined with calcium oxide (CaO) to form CFO-1113, calcium chloride, and water, as shown in reaction (3) below:

2CHClF—CClF₂+CaO→CF₂═CFCl+CaCl₂+H₂O (3)

HCFC-123a can be combined with calcium hydroxide (Ca(OH)₂) to form CFO-1113, calcium chloride, and water, as shown in reaction (4) below:

2CHClF—CClF₂+Ca(OH)₂→CF₂═CFCl+CaCl₂+2H₂O (4)

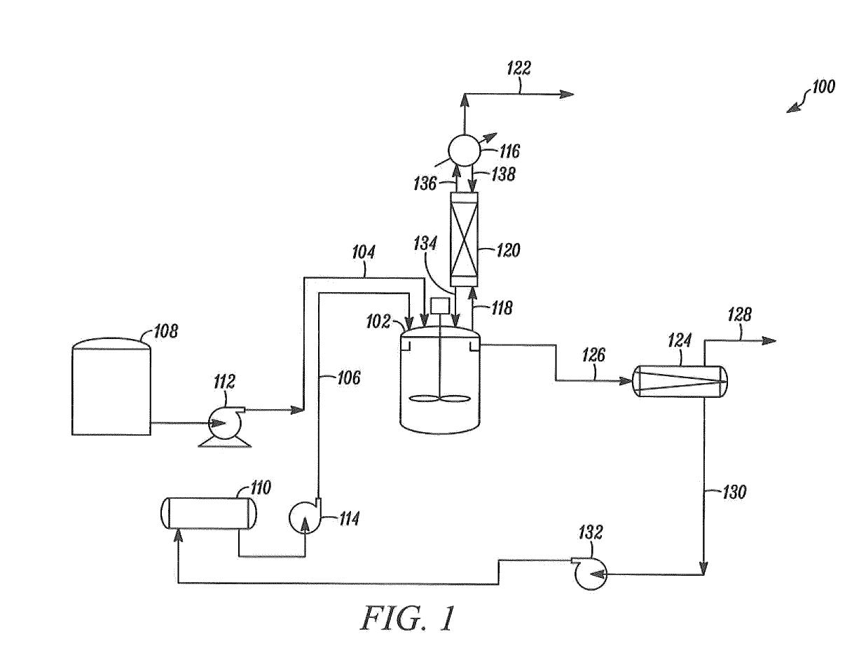

FIG. 1 illustrates one system for converting HCFC-123a to CFO-1113. The system 100 includes at least one reactor 102. The reactor 102 as illustrated in FIG. 1 is a continuous stirred tank reactor. The reactor 102 can alternatively be any other suitable type of reactor, including, but not limited to, a batch reactor, or a semi-continuous reactor. Accordingly, the reaction in the at least one reactor can be run continuously, in batch mode, or in semi-continuous mode. In examples where reactor 102 is a batch reactor, the reaction can proceed until either the HCFC-123a or the KOH is consumed. The batch reactor can then be shut down, cleaned out, which can include stopping the reaction in the batch reactor to remove salt and/or salt solution, and then restarted for another round of the reaction. In examples where the reactor 102 is a continuous stirred tank reactor, the reaction can be maintained in a continuous manner, and frequent startups and shutdowns of the reactor can be avoided. In at least some examples, use of a continuous stirred tank reactor can allow for smaller equipment as compared to a batch reactor, which can be more economical.

As illustrated in FIG. 1, the system 100 for converting HCFC-123a to CFO-1113 includes a first feed line 104 and a second feed line 106. A base containing feed stream can be fed to the reactor 102 through the first feed line 104, and an HCFC-123a containing feed stream can be fed to the reactor 102 through the second feed line 106. As illustrated, the base containing feed stream can be provided to the first feed line 104 from a first storage tank 108, and the HCFC-123a containing feed stream can be provided to the second feed line 106 from a second storage tank 110. At least one pump 112 can be utilized to provide the base containing feed stream from the first storage tank 108 to the reactor 102 through the first feed line 104. Similarly, at least one pump 114 can be utilized to provide the HCFC-123a containing feed stream from the second storage tank 110 to the reactor 102 through the second feed line 106.

In accordance with the system 100 illustrated in FIG. 1, a process for converting HCFC-123a to CFO-1113 can include providing at least one reactor 102, providing an HCFC-123a containing feed stream to the reactor 102, and providing a base containing feed stream to the reactor 102. The HCFC-123a containing feed stream and the base containing feed stream can be provided to the reactor 102 continuously. In one example, the HCFC-123a containing feed stream and the base containing feed stream can be provided to the reactor 102 in rates and amounts that result in a mole ratio of base to HCFC-123a in the reactor 102 of less that about 0.5:1 up to about to 3:1, preferably from about 1:1 to about 1.5:1. The base containing feed stream can be an aqueous mixture, and can contain the base at any suitable strength for producing the desired reaction. For example, when KOH is used as the base, the base containing feed stream can contain base at a strength from about 5 wt % to about 50 wt %, preferably from about 20 wt % to about 40 wt %. Optionally, a phase transfer agent or catalyst, such as, for example, alcohol and Aliquat®336 (C₂₅H₅₄ClN) can be added to the reaction in the at least one reactor 102, which can enhance the reaction rate.

In the reactor 102, the HCFC-123a and the base can be reacted at any suitable temperature, preferably at a temperature from about 40° C. to about 100° C., and more preferably at a temperature from about 50° C. to about 90° C. Reacting the HCFC-123a and base in the reactor 102 forms reaction product vapors, which can include CFO-1113, HCFC-123a, water, and other byproducts.

As illustrated in FIG. 1, the system 100 also includes a condenser 116. Condenser 116 can receive the reaction product vapors formed during the reaction that occurs in the reactor 102. The reaction product vapors formed during the reaction that occurs in the reactor 102 can include CFO-1113, HCFC-123a, and water vapor. The reaction product vapors can also contain at least some entrained KOH and KCl.

The reaction product vapor stream can be provided to condenser 116 through one or more lines, such as lines 118 and 136. The condenser 116 can separate CFO-1113 from the reaction product vapors, and a CFO-1113 product stream can be removed from the condenser 116. As illustrated in FIG. 1, the CFO-1113 product stream can be removed from the condenser 116 through line 122. A return stream including condensed HCFC-123a and water can be returned to the reactor 102 via one or more lines, such as lines 134 and 138.

The system 100 can also optionally include a fractional distillation column 120. As illustrated in FIG. 1, the fractional distillation column 120 is located between the reactor 102 and the condenser 116. The fractional distillation column 120 can receive the reaction product vapor stream through line 118. The fractional distillation column 120 can enhance separation of CFO-1113 from the reaction vapor product stream that is removed from the reactor 102. HCFC-123a, water, and any other products separated from the CFO-1113 in the fractional distillation column 120 can be returned to the reactor 102. Additionally, the return stream from the condenser can be received by the fractional distillation column 120 through line 138, and can be returned through line 134 to the reactor 102 along with the products separated from the CFO-1113 in the fractional distillation column 120.

In examples where the reaction of HCFC-123a and the base is conducted as a continuous reaction in reactor 102, a liquid effluent can be removed from the reactor 102 either continuously or periodically. The liquid effluent can be removed from the reactor 102 through line 126. The liquid effluent can include base, water, unreacted HCFC-123a, and byproducts of the reaction. The liquid effluent can be passed to a phase separator 124. The phase separator 124 can receive the liquid effluent and separate the unreacted HCFC-123a from the other components of the liquid effluent stream. An unreacted HCFC-123a stream can be removed from the phase separator 124 through line 130, and the remaining components of the liquid effluent stream can be removed from the phase separator 124 in a spent product stream through line 128. As illustrated in FIG. 1, at least one pump 132 can be utilized to pump the unreacted HCFC-123a to second storage tank 110. Alternatively, the unreacted HCFC-123a can be removed from the phase separator 124 and can be provided directly to the reactor 102, to a different storage tank, or to some other process unit.

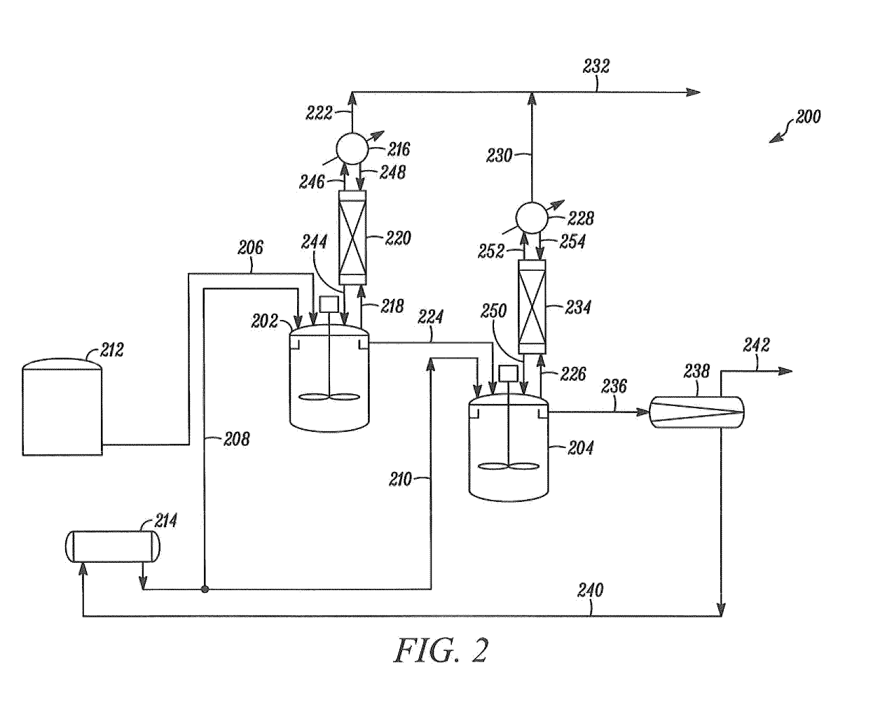

FIG. 2 illustrates a second example of a system for converting HCFC-123a to CFO-1113, illustrated generally at 200. The system 200 includes a plurality of reactors in series. As illustrated, system 200 includes at least a first reactor 202 and a second rector 204. It should be understood that, although two reactors are shown for illustrative purposes in FIG. 2, the system 200 can include any suitable number of reactors, including more than two reactors. Each of the reactors 202 and 204 as illustrated in FIG. 2 is a continuous stirred tank reactor. The reactors 202 and 204 can alternatively be any other suitable type of reactor, including, but not limited to, a batch reactor, or a semi-continuous reactor, as discussed above with respect to FIG. 1. Accordingly, the reaction in the at least one reactor can be run continuously, in batch mode, or in semi-continuous mode. In examples where the reactors 202 and 204 are both continuous stirred tank reactors, the reaction can preferably be maintained in a continuous manner.

As illustrated in FIG. 2, the system 200 for converting HCFC-123a to CFO-1113 includes a first feed line 206, a second feed line 208, and a third feed line 210. A base containing feed stream can be fed to the first reactor 202 through the first feed line 206. An HCFC-123a containing feed stream can optionally be fed to the first reactor 202 through the second feed line 208, and can optionally be fed to the second reactor 204 through the third feed line 210. As illustrated, the base containing feed stream can be provided to the first feed line 206 from a first storage tank 212. The HCFC-123a containing feed stream can be provided to the first reactor 202 through the second reactor line 208 from a second storage tank 214. The HCFC-123a containing feed stream can also be provided from the second storage tank 214 to the second reactor 204 through the third feed line 210. Although not illustrated in FIG. 2, it should be understood that pumps can be utilized to with the reactor feed lines 206, 208 and 210 in the same manner as described above with respect to reactor feed lines 104 and 106.

In the system illustrated in FIG. 2, the reaction for converting HCFC-123a to CFO-1113 can be conducted simultaneously in the plurality of reactors. In at least one example, the reaction is conducted simultaneously and continuously in the first reactor 202 and the second reactor 204. In each of the reactors 202 and 204, HCFC-123a and base can be reacted at any suitable temperature, preferably at a temperature from about 40° C. to about 100° C., and more preferably at a temperature from about 50° C. to about 90° C. Reacting the HCFC-123a and the base in the reactors 202 and 204 forms reaction product vapors, which can include CFO-1113, HCFC-123a, water, and other byproducts. Optionally, a phase transfer agent or catalyst, such as, for example, alcohol and Aliquat®336 (C₂₅H₅₄ClN) can be added to the reaction in either or both of the reactors 202 and 204, which can enhance the reaction rate.

In the system illustrated in FIG. 2, the process for converting HCFC-123a to CFO-1113 can be conducted with respect to reactor 202 in a manner similar to that discussed above with respect to reactor 102. The base containing feed stream can be provided to the first reactor 202, and an HCFC-123a containing feed stream can also be provided to the first reactor 202. The HCFC-123a containing feed stream and the base containing feed stream can be provided to the reactor 202 continuously. In one example, the HCFC-123a containing feed stream and the base containing feed stream can be provided to the first reactor 202 in rates and amounts that result in a mole ratio of base to HCFC-123a in the first reactor 202 of less that about 0.5:1 up to about to 3:1, preferably from about 1:1 to about 1.5:1.

Reaction product vapors can be removed from the first reactor 202. The reaction product vapors can be provided directly to a first condenser 216 through one or more lines, such as lines 218 and 246. First condenser 216 can receive the reaction product vapors formed during the reaction that occurs in the first reactor 202. The first condenser 216 can separate CFO-1113 from other less volatile components of the reaction product vapors, and a CFO-1113 product stream can be removed from the first condenser 216. As illustrated in FIG. 2, the CFO-1113 product stream can be removed from the first condenser 216 through line 222. A return stream including condensed HCFC-123a and water can be returned to the first reactor 202 through one or more lines, such as lines 248 and 244.

The system 200 can also optionally include a first fractional distillation column 220. As illustrated in FIG. 2, the first fractional distillation column 220 is located between the first reactor 202 and the first condenser 216. The first fractional distillation column 220 can receive the reaction product vapor stream from the first reactor 202 through line 218. The first fractional distillation column 220 can enhance separation of CFO-1113 from the reaction vapor product stream that is removed from the first reactor 202. HCFC-123a, water, and any other products separated from the CFO-1113 in the first fractional distillation column 220 can be returned to the first reactor 202 via line 244. Additionally, the return stream from the first condenser 216 can be received by the first fractional distillation column 220 through line 248, and can be returned through line 244 to the first reactor 202 along with the products separated from the CFO-1113 in the first fractional distillation column 220. Alternatively, the return streams from the first condenser 216 and the first fractional distillation column 120 can be provided to a downstream reactor, such as the second reactor 204.

In examples where the reaction of the HCFC-123a and the base is conducted as a continuous reaction in the first reactor 202, a liquid effluent can be removed from the first reactor 202 through line 224. The liquid effluent is preferably removed continuously, and can be fed to the second reactor 204. The liquid effluent can include base, water, unreacted HCFC-123a, and other byproducts. The liquid effluent stream provided from the first reactor 202 to the second reactor 204 is thus a feed stream that contains both HCFC-123a and base. The HCFC-123a and the base can be reacted in the second reactor 204 to form reaction product vapors. The reaction product vapors formed in the second reactor 204 can include CFO-1113, HCFC-123a, water, and other byproducts.

Optionally, additional HCFC-123a can be provided to the second reactor 204 by providing an HCFC-123a containing feed stream to the second reactor that is separate from the liquid effluent. The separate HCFC-123a containing feed stream can be fed to the second reactor 204 through third feed line 210. In such an example, the HCFC-123a included in the liquid effluent stream from the first reactor 202 and the HCFC-123a containing feed stream fed to the second reactor 204 can be reacted with the base in the liquid effluent stream to form the reaction product vapors. The HCFC-123a containing feed stream can be provided to the second reactor 204 at a rate or amount appropriate to supplement the HCFC-123a in the liquid effluent from the first reactor 202, to provide a total amount of HCFC-123a in the second reactor 204 that results in a mole ratio of base to HCFC-123a in the second reactor 204 of less that about 0.5:1 up to about to 3:1, preferably from about 1:1 to about 1.5:1. The HCFC-123a and base can be reacted in the second reactor 204 at any suitable temperature, preferably at a temperature from about 40° C. to about 100° C., and more preferably at a temperature from about 50° C. to about 90° C.

The reaction product vapors produced in the second reactor 204 can be removed from the second reactor 204. The reaction product vapors can be provided directly to a second condenser 228, which can receive the reaction product vapors formed during the reaction that occurs in the second reactor 204 through one or more lines, such as lines 226 and 252. The second condenser 228 can separate CFO-1113 from other less volatile components of the reaction product vapors, and a CFO-1113 product stream can be removed from the second condenser 228. As illustrated in FIG. 2, the CFO-1113 product stream can be removed from the second condenser 228 through line 230. The CFO-1113 product stream from the second reactor 204 can be combined with the CFO-1113 product stream from the first reactor 202, and can be passed downstream through a line 232. A return stream including condensed HCFC-123a and water can be returned to the second reactor 204 via one or more lines, such as lines 254 and 250.

The system 200 can also optionally include a second fractional distillation column 234. As illustrated in FIG. 2, the second fractional distillation column 234 is located between the second reactor 204 and the second condenser 228. The second fractional distillation column 234 can receive the reaction product vapor stream from the second reactor 204 through line 226. The second fractional distillation column 234 can enhance separation of CFO-1113 from the reaction vapor product stream that is removed from the second reactor 204. HCFC-123a, water, and any other products separated from the CFO-1113 in the second fractional distillation column 234 can be returned to the second reactor 204 through line 250. Additionally, the return stream from the second condenser 228 can be received by the second fractional distillation column 234 through line 254, and can be returned to the second reactor 204 through line 250 along with the products separated from the CFO-1113 in the second fractional distillation column 234.

In examples where the reaction of HCFC-123a and base is conducted as a continuous reaction in the second reactor 204, a liquid effluent can be removed from the second reactor 204 through line 236 either continuously or periodically. The liquid effluent can include base, water, unreacted HCFC-123a, and other byproducts.

As illustrated in FIG. 2, the liquid effluent removed from the second reactor 204 can be passed to a phase separator 238. Alternatively, in systems that include a greater plurality of reactors, the liquid effluent removed from the second reactor 204 could be provided to a downstream reactor. In the illustrated embodiment, the phase separator 238 can receive the liquid effluent from the second reactor 204, and can separate the unreacted HCFC-123a from the other components of the liquid effluent stream. An unreacted HCFC-123a stream can be removed from the phase separator 238 through line 240, and the remaining components of the liquid effluent stream can be removed from the phase separator 238 in a spent product stream through line 242. As illustrated in FIG. 2, the unreacted HCFC-123a can be provided to the second storage tank 214. Alternatively, the unreacted HCFC-123a can be removed from the phase separator 238 and can be provided directly to the first reactor 202, the second reactor 204, to a different storage tank, or to some other process unit.

### EXAMPLE 1

A reaction for converting HCFC-123a to CFO-1113 was carried out in a system having a single reactor. The reactor was a continuous stirred reactor. The reaction was conducted continuously for 52 (fifty-two) hours. During the experiment, the temperature in the reactor varied between about 50° C. and about 60° C. The pressure in the reactor varied between about 30 psig and 113 psig.

During the reaction, an organic feed stream containing HCFC-123a was provided to the reactor at a flow rate of about 5 ml/min. Gas chromatography area percents of the organic feed revealed that the organic feed contained about 0.81% HFO-1113, about 95% HCFC-123a, and about 3.2% CFC-113.

During the reaction, a KOH feed stream was provided to the reactor at a flow rate of about 12 ml/min. Based upon the feed rates of the organic feed stream and the KOH feed stream, the molar ratio of KOH to HCFC-123a was calculated to be about 1, or slightly above 1. The theoretical residence time in the reactor during the reaction was calculated to be about 2 hours.

A collection vessel was used to collect spent KOH and any organic that was carried out with spent KOH. MeCl2 was added inside the collection vessel in order to trap organic. The volume of MeCl2 inside the cylinder was 20% of the total volume of liquid in collection vessel. Product and un-reacted organic was collected in collection vessel that were held in dry ice. Vapor samples were taken once an hour, and liquid samples were taken at longer intervals.

The overall mass balance of the reaction was calculated to be about 82% for HCFC-123a and about 87% for KOH. The average single pass conversion was calculated to be about 39%. Selectivity to CFO-1113 was calculated to be about 90%.

### EXAMPLE 2

Simulation and methods known to those skilled in the art were utilized to generate the following example utilizing a plurality of reactors in series to convert HCFC-123a to CFC-1113 by reacting the HCFC-123a with KOH. Using data obtained from Example 1, a two stage system was simulated. The two stage system included a first reactor and a second reactor, as described with respect to FIG. 2 above. In the simulation, the first reactor was operated at the same feed conditions as the reactor in Example 1. A lower conversion was utilized in the second reactor to compensate for the dilution effect in second reactor, although it is contemplated that operation at a higher temperature in second reactor could be utilized to maintain the same conversion as the first reactor.

By utilizing the same residence time in each of the two reactors, an the overall of conversion of HCFC-123a across two reactors was calculated to be about 56%. A corresponding increase in KOH utilization was also calculated.

From the foregoing, it will be appreciated that although specific examples have been described herein for purposes of illustration, various modifications may be made without deviating from the spirit or scope of this disclosure. It is therefore intended that the foregoing detailed description be regarded as illustrative rather than limiting, and that it be understood that it is the following claims, including all equivalents, that are intended to particularly point out and distinctly claim the claimed subject matter.

What is claimed is:

1. A system for producing CFO-1113 from HCFC-123a, the system comprising:
at least one reactor that receives an HCFC-123a containing feed stream and a base containing feed stream, wherein the HCFC-123a and the base are reacted in the at least one reactor to produce reaction product vapors including CFO-1113, and a liquid effluent stream containing unreacted HCFC-123a is removed from the at least one reactor;
a condenser that receives reaction product vapors from the reactor and produces a CFO-1113 product stream; and
a phase separator that receives a liquid effluent stream from the reactor and separates unreacted HCFC-123a to produce an unreacted HCFC-123a stream.

2. The system of claim 1, wherein the system comprises a plurality of reactors, including at least a first reactor and a second reactor.

3. The system of claim 2, wherein the base containing feed stream containing that is received by the second reactor is the liquid effluent stream that is removed from the first reactor.

4. The system of claim 1, wherein the HCFC-123a and the base are reacted in the at least one reactor at a temperature from about 40° C. to about 100° C.

5. The system of claim 1, wherein the base is selected from the group consisting of potassium hydroxide (KOH), sodium hydroxide (NaOH), calcium hydroxide (Ca(OH)₂), and Calcium oxide (CaO).

6. The system of claim 1, wherein the mole ratio of base to HCFC-123a in the at least one reactor is from less than about 0.5:1 up to about 3:1.

7. The system of claim 1, wherein a phase transfer agent or catalyst is added to the reaction in the at least one reactor.

8. The system of claim 1, further comprising a fractional distillation column between the at least one reactor and the condenser.

9. The system of claim 1, wherein the reaction in the at least one reactor is conducted continuously.

10. The system of claim 1, wherein the reaction in the at least one reactor is conducted in batch mode.

11. The system of claim 1, wherein the reaction in the at least one reactor is conducted in semi-continuous mode.

12. A process for producing CFO-1113 from HCFC-123a comprising the steps of:
providing a first reactor;
providing an HCFC-123a containing feed stream to the first reactor;
providing a base containing feed stream to the first reactor;
reacting the HCFC-123a and the base in the first reactor to produce reaction product vapors including CFO-1113;
removing the reaction product vapors from the first reactor; and
removing a liquid effluent stream from the first reactor, where the liquid effluent stream contains base and unreacted HCFC-123a.

13. The process of claim 12, wherein the HCFC-123a and the base are reacted continuously.

14. The process of claim 12, wherein the HCFC-123a and the base are reacted in the at least one reactor at a temperature from about 40° C. to about 100° C.

15. The process of claim 12, wherein the base is selected from the group consisting of potassium hydroxide (KOH), sodium hydroxide (NaOH), calcium hydroxide (Ca(OH)₂), and Calcium oxide (CaO).

16. The process of claim 12, wherein the mole ratio of base to HCFC-123a in the at least one reactor is from less than about 0.5:1 up to about 3:1.

17. The process of claim 12, wherein a phase transfer agent or catalyst is added during the step of reacting.

18. The process of claim 12, further comprising the steps of:
separating CFO-1113 from the reaction product vapors to produce a CFO-1113 containing product stream.

19. The process of claim 12, further comprising the steps of:
providing a second reactor;
providing the liquid effluent stream containing base and unreacted HCFC-123a from the first reactor to the second reactor;
optionally providing additional HCFC-123a to the second reactor;
reacting the HCFC-123a and the base in the second reactor to produce reaction product vapors including CFO-1113;
removing the reaction product vapors from the second reactor; and
removing a liquid effluent stream from the second reactor, where the liquid effluent stream contains base and unreacted HCFC-123a.

20. The process of claim 19, wherein the base is selected from the group consisting of potassium hydroxide (KOH), sodium hydroxide (NaOH), calcium hydroxide (Ca(OH)₂), and calcium oxide (CaO).

21. The process of claim 19, wherein the mole ratio of base to HCFC-123a in the second reactor is from less than about 0.5:1 up to about 3:1.

22. A process for producing CFO-1113 from HCFC-123a comprising the steps of:
providing a first reactor;
providing an HCFC-123a containing feed stream to the first reactor;
providing a base containing feed stream to the first reactor;
reacting the HCFC-123a and the base in the first reactor to produce reaction product vapors including CFO-1113;
removing the reaction product vapors from the first reactor;
removing a liquid effluent stream from the first reactor, where the liquid effluent stream contains base and unreacted HCFC-123a;
providing a second reactor;
providing the liquid effluent stream containing base and unreacted HCFC-123a from the first reactor to the second reactor;
optionally providing additional HCFC-123a to the second reactor;
reacting the HCFC-123a and the base in the second reactor to produce reaction product vapors including CFO-1113;
removing the reaction product vapors from the second reactor; and
removing a liquid effluent stream from the second reactor, where the liquid effluent stream contains base and unreacted HCFC-123a.
